(19) United States
(12) Patent Application Publication
(10) Pub. No.: US 2021/0346178 A1
(43) Pub. Date: Nov. 11, 2021

(54) METHOD FOR USER-SIDE VOLUME ADAPTATION OF A BREAST PROSTHESIS

(71) Applicant: AMOENA MEDIZIN-ORTHOPAEDIE-TECHNIK GMBH, Raubling (DE)

(72) Inventors: Nils STELTER, Frasdorf (DE); Helmut WILD, Stephanskirchen (DE)

(21) Appl. No.: 17/285,867

(22) PCT Filed: Oct. 17, 2019
(86) PCT No.: PCT/EP2019/078259

§ 371 Date: Apr. 15, 2021

(30) Foreign Application Priority Data
Oct. 19, 2018 (DE) 10 2018 126 035.0

(51) Int. Cl. A61F 2/52

(52) U.S. Cl. CPC A61F 2/52; A61F 2002/501

(57) ABSTRACT

The present disclosure relates to a method for volume adaptation of a breast prosthesis, wherein the breast prosthesis comprises a first shell body, a second shell body connected circumferentially thereto, and a fluid chamber arranged between the shell bodies. The fluid chamber is accessible from the outside by way of a passage provided having a valve, wherein the method comprises (a) inserting the breast prosthesis into a brassiere worn by a patient, and (b) filling the fluid space with a fluid or removing a fluid from the fluid space while the breast prosthesis is inserted into the brassiere worn by the patient.

The invention relates to a method for a user side volume adaptation of a breast prosthesis.

Breast prostheses are worn after surgical breast removals. Demands on breast prostheses in particular include a shape and feel that come as close as possible to the natural breast as well as a high comfort in wear.

To be able to accomplish being able to adapt the volume of such a prosthesis to the individual needs of the wearer that result from the size of the still healthy breast, if present, or from personal well-being without expensive custom-made solutions, it has already been proposed in the prior art to provide breast prostheses whose volume can be retroactively adapted. EP 0 824 001 A2 is to be named as an example for this.

It is the object of the invention to provide a method for a user-side volume adaptation of a breast prosthesis suitable for this purpose.

Against this background, the invention relates to a method for the volume adaptation of a breast prosthesis, wherein the breast prosthesis has a first shell body, a second shell body peripherally connected thereto, and a fluid space arranged between said shell bodies, wherein the fluid space is accessible from the outside through a passage provided with a valve, the method comprising the following steps: (a) inserting the breast prosthesis into a brassiere worn by a patient; (b) filling the fluid space with a fluid or removing a fluid from the fluid space while the breast prosthesis is inserted into the brassiere worn by the patient.

The shell bodies of the breast prosthesis used in accordance with the invention are preferably film bags filled with a deformable material. The deformable material is preferably a crosslinked silicone rubber, preferably a two-component silicone rubber. The films of the film bag are preferably plastic films. At least four films are preferably connected, in particular welded, along a common periphery. This design allows the shell bodies to satisfy the demands on feel and on comfort in wear and not to impede an expansion of the fluid space volume on its filling with a fluid or a shrinking of the fluid space volume on the removal of fluid.

The silicone rubber of the shell bodies can comprise additional components such as a phase change material or a porous granulate or hollow spheres. Phase change materials serve the improvement of heat regulation at the skin of the wearer and are therefore preferably admixed to the silicone rubber of the first shell body that should lie on the side of the wearer in use. Suitable phase change materials include those whose phase transition temperature is close to body temperature. Examples include paraffins having a suitable number of carbon atoms, typically approximately twenty, to set a melting point in the desired range. Granulates or hollow spheres can serve to reduce the weight of the prosthesis without impairing the haptic properties.

The fluid for the volume adaptation can be a gas, in particular air. A simple air pump can be used for the volume adaptation in this embodiment.

The fluid for the volume adaptation can alternatively be a liquid, in particular a subsequently crosslinkable viscous liquid. A particularly suitable example includes a crosslinkable silicone fluid. In this case, the method can furthermore include a step of the subsequent crosslinking of the liquid filled in the fluid space. It can be achieved by the use and subsequent crosslinking that the filling of the hollow space with the shell bodies has comparable physical properties, which can have a positive effect on the haptic properties of the prosthesis overall. The step of the subsequent crosslinking takes place in a few minutes at room temperature in the brassiere. It must be noted that a crosslinked fluid can no longer be subsequently removed to reduce the volume.

Combinations of gas and liquid can also be used as a fluid for volume adaptation in an embodiment of the invention.

Provision can be made as part of step (a) that the volume-adaptable breast prosthesis is placed between the skin of the patient and the cup of the brassiere. in the case of a brassiere having integrated pockets, provision can alternatively be made that the breast prosthesis is placed into the respective pocket of the brassiere as part of step (a).

Provision is made in an embodiment that an additional medium is present within the fluid space that is different from the fluid for volume adaptation to be filled in step (b) and that reduces the adhesive tendency of the oppositely disposed inner surfaces of the shell bodies. The subsequent volume adaptation in accordance with the invention can be impeded in that the inner surfaces of the shell bodies adhere to one another. A permanent deformation of the breast prosthesis by such an adhesion would also have to be feared. The tendency for such an unwanted adhesion is reduced by the additional medium.

The medium for reducing the adhesive tendency of the oppositely disposed inner surfaces of the shell bodies can be a liquid, in particular an oil, and further preferably a silicone oil. In the event that both the fluid for the volume adaptation and the medium for reducing the adhesive tendency of the oppositely disposed inner surfaces of the shell bodies are respectively liquids, these liquids differ from one another.

The medium for reducing the adhesive tendency of the oppositely disposed inner surfaces of the shell bodies can furthermore be a powdery solid. The average particle size of the powder grains is preferably in the range between 1 nm to 1 mm.

The medium for reducing the adhesive tendency of the oppositely disposed inner surfaces of the shell bodies can optionally also be a gas. In the event that both the fluid for the volume adaptation and the medium for reducing the adhesive tendency of the oppositely disposed inner surfaces of the shell bodies are respectively gases, these gases differ from one another.

Combinations of said media for reducing the adhesive tendency of the oppositely disposed inner surfaces of the shell bodies are also used in further embodiments. A mixture of liquids and dispersed powdery solids can be used, for example, with the liquids and dispersed powdery solids being able to be formed as defined above.

The passage can be a valve tube that preferably extends in a radial direction through the connection region between the two shell bodies. The tube is preferably adhesively bonded or welded between the film bags and thus passes through the weld seam or adhesive seam between the film bags in the radial direction. The valve tube preferably comprises a flexible material and in particular a flexible plastic material. Provision can be made that the tube projects beyond the connection region into the fluid space. The valve tube generally has a check valve such as a flutter valve.

Provision can be made that a needle is placed into the valve tube to fill or empty the fluid space and that the fluid for volume adaptation is pressed through this needle into the fluid space. The needle can be produced from plastic or metal, for example. The needle is preferably blunt in order not to damage the valve, for example the membrane of the flutter valve.

Provision is made in a variant that the valve tube comprises a step at its inner side at which its inner diameter reduces in the direction toward the fluid space and that the needle preferably has a corresponding step at the outer side at which its inner diameter reduces in the direction toward the fluid space. Each of the steps can individually have a straight step surface, i.e. a step surface standing normally on the tube axis or needle axis, or an inclined step surface, i.e. a step surface standing at a slant to the tube axis or needle axis. The corresponding steps can serve as abutments to prevent too deep a penetration of the needle.

The breast prosthesis can be connected to a filling and/or removal device between steps (a) and (b) as part of the method. The filling and/or removal device can have a pump, a tube, and a needle, for example. In an embodiment, the breast prosthesis can therefore already be connected to a filling and/or removal device before it is placed into the brassiere. Provision can alternatively be made that the breast prosthesis is only connected to a filling and/or removal device after it has been placed into the brassiere.

The filling and/or removal device can be connected to the brassiere and/or to the patient before or after it is connected to the breast prosthesis. Provision can, for example be made that that the filling and/or removal device is clamped at a suitable point between a strap of the brassiere and the skin of the patient to hold it in a suitable position on the insertion of the prosthesis and optionally also already on the connection of the prosthesis that has not yet been inserted.

The volume of only one breast prosthesis can be adapted as part of the present method. The breast prosthesis is placed on a side of the brassiere for this purpose and its size is adapted by filling or emptying the fluid space until its size corresponds to the size of the other, for example healthy, breast.

Provision can also be made that the volumes of two breast prostheses are adapted in the course of the present method, for example in the case of two amputated breasts or of a size change desired by the wearer. In this case, breast prostheses are placed into both sides of the brassiere and their sizes are adapted after one another, optionally in a plurality of sequences, by filling or emptying the fluid space.

Further details and advantages of the invention result from the embodiments described in the following with reference to the Figures. There are shown in the Figures:

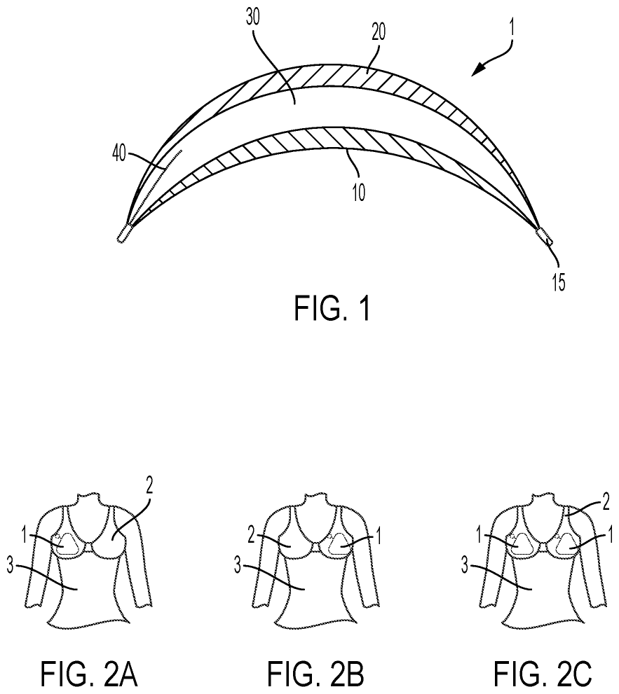

FIG. 1: a schematic representation of a volume-adaptable breast prosthesis usable as part of the method in accordance with the invention;

FIG. 2: a schematic representation of a patient with a brassiere and a breast prosthesis on the right side, on the left side, and in both sides of the brassiere;

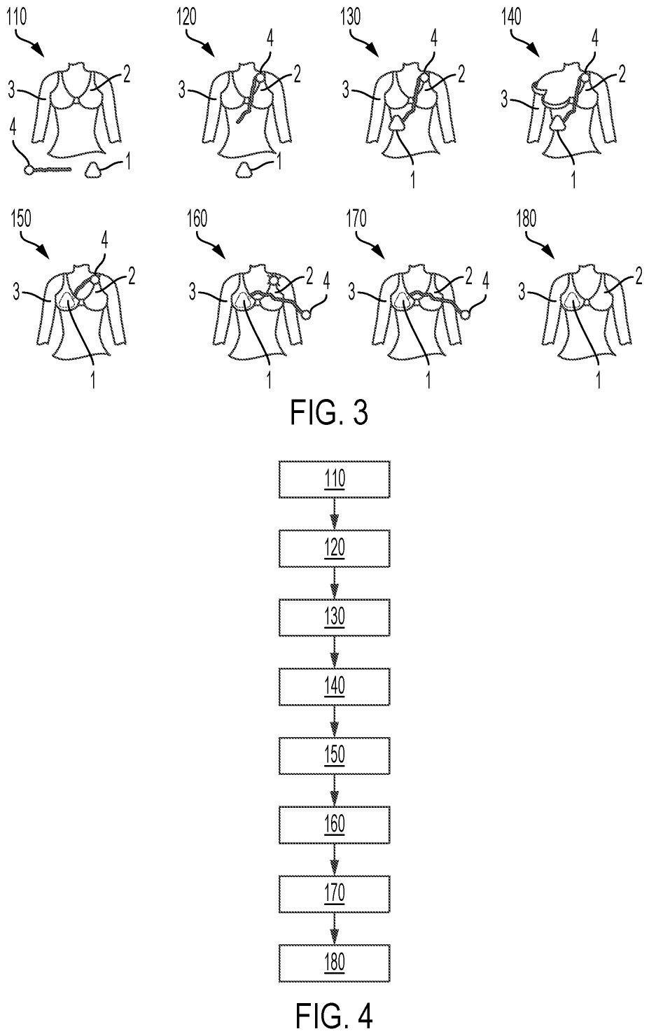

FIG. 3: a figurative representation of a procedure of an embodiment variant of a method in accordance with the invention; and

FIG. 4: an associated flowchart;

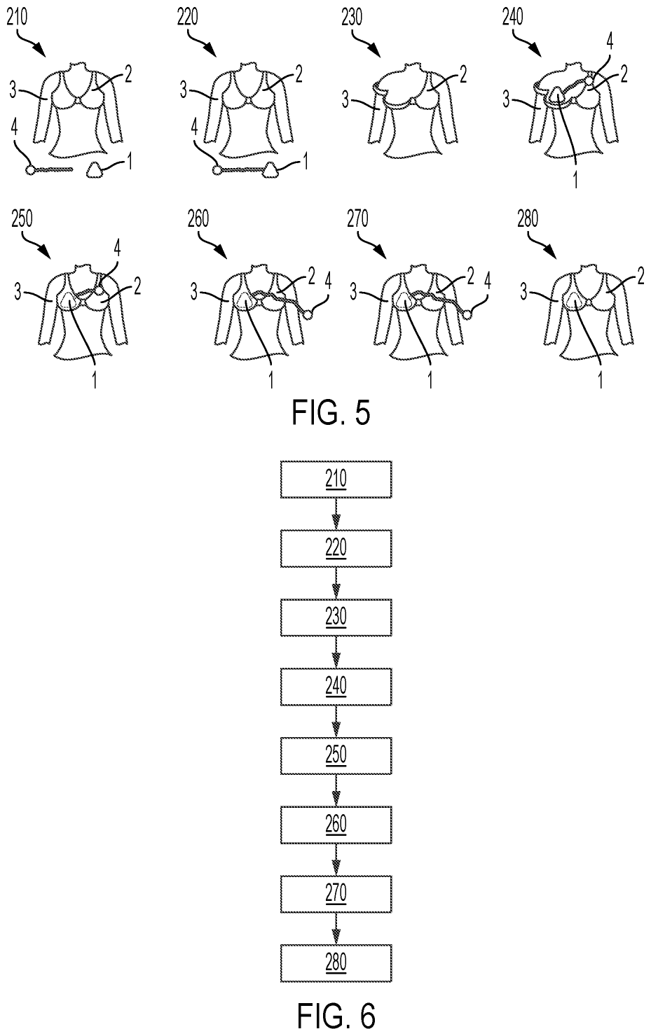

FIG. 5: a figurative representation of a procedure of a further embodiment variant of a method in accordance with the invention; and

FIG. 6: an associated flowchart.

The breast prosthesis 1 having an adaptable volume shown in FIG. 1 can be used as part of a method in accordance with the invention. The breast prosthesis 1 comprises a first shell body 10 at the lower side of the prosthesis 1 facing the wearer and a second shell body 20 peripherally connected thereto and at the upper side of the prosthesis 1 facing away from the wearer. Both shell bodies 10 and 20 are film bags that are fill with a crosslinked two-component silicone rubber compound. The film bags are each produced from two plastic film pieces that are welded to one another along the common peripheral surface.

The shell bodies 10 and 20 are in turn connected along a peripheral weld seam 15 such that a fluid space 30 is formed between them that can, for example, be filled with air, but also with a liquid. The volume of the breast prosthesis 1 can be adapted by filling and emptying the fluid space 30.

To make a subsequent filling and emptying of the fluid space 30 possible, i.e. one taking place after the production, the breast prosthesis 1 comprises a valve tube 40 that is composed of a flexible plastic material, that comprises a flat flutter valve, that penetrates the weld seam 15 in the radial direction, and that is welded between the shell bodies 10 and 20. The valve tube 40 does not only reach up to the end of the weld seam 15, but projects freely, i.e. without being connected to one of the shell bodies 10 or 20, beyond the weld seam 15 into the fluid space 30. The section of the valve tube 40 projecting into the fluid space 30 is therefore freely movable in the fluid space 30.

To prevent a sticking together of the plastic inner surfaces of the two shell bodies 10 and 20 due, for example, to electrostatic interaction and thus an unwanted adhesion of the fluid space 30, the fluid space 30 is filled with a smaller amount of silicone oil.

FIG. 2 shows a basic procedure of a method in accordance with the invention for volume adaptation at the right (FIG. 2a), left (FIG. 2b), and both sides (FIG. 2c) by means of a fluid of one or two breast prostheses 1. Provision is made here that the volume-adaptable breast prosthesis 1 can be placed between the skin and the cup of the brassiere 2. If the brassiere 2 has integrated pockets, the prosthesis 1 can also be placed therein. The volume of the volume-adaptable prosthesis 1 can be continuously increased or decreased in size in this position as part of a method in accordance with the invention by the user 3 or by a further person until the desired volume is reached, for instance based on the feeling of the user 3.

FIGS. 3-4 show a figurative representation of a procedure of an embodiment variant of a method in accordance with the invention and an associated flowchart. In a first step 110, the user 3 who is wearing a brassiere 2, arranges the volume-adaptable breast prosthesis 1 and a suitable pump accessory 4, for example comprising a pump, a tube, and a needle. In a second step 120, the pump accessory 4 is clamped at a suitable position between the brassiere 2 and the skin of the patient 3, for example between the straps of the brassiere 2 and the skin of the patient 3. In a further step 130, the free end of the pump accessory 4, that is, for example, the needle, is then connected to the volume-adaptable breast prosthesis 1 in that, for example, the needle is placed into the valve tube. In a next step 140, the brassiere 2 is opened and the breast area exposed at the side of the brassiere 2 at which the breast prosthesis 1 should be inserted (in this case at the right from the viewpoint of the wearer). The volume-adaptable breast prosthesis 1 is then placed between the skin 3 of the patient and the cup of the brassiere 2 in step 150. In the case of a brassiere 2 having integrated pockets, the breast prosthesis 1 can also be placed therein. The brassiere is then closed again and brought into the normal position of wear. The clamped region of the pump accessory 4 is subsequently removed using a free hand of the user 3 in a step 160. In step 170, the user 3 then increases or reduces the volume of the breast prosthesis by means of the pup accessory 4. The method in accordance with the invention ends at step 180 with the finalizing of the increasing or decreasing procedure when the desired size has been reached.

FIGS. 5-6 show a figurative representation of a procedure of a further embodiment variant of a method in accordance with the invention and an associated flowchart. In a first step 210 of this embodiment variant, the user 3 who is wearing a suitable brassiere 2, arranges the volume-adaptable breast prosthesis 1 and a suitable accessory 4 comprising a pump, a tube, and a cannula. In second step 220, the user 3 connects the pump accessory 4 to the breast prosthesis 1, for example by inserting the needle into the valve tube. In a subsequent step 230, the brassiere 2 is then opened at the side (at the right from the viewpoint of the wearer 3) at which the breast prosthesis 1 is to be inserted and the breast area is exposed. As part of step 240, the free end of the pump accessory 4 is then fastened at a suitable position between the brassiere 2 and the skin of the patient 3, for example clamped between the straps of the brassiere 2 and the skin of the patient 3. The breast prosthesis 1 is placed onto the free breast side before this, after this, or simultaneously therewith. In step 250, the brassiere 1 is then closed again and brought into the normal position of wear. The following steps 260-280 correspond to steps 160-180 of the embodiment in accordance with FIGS. 3-4.

The volume adaptation by means of the suitable pump accessory 4 can take place as part of both method procedures shown, for example also by the user 3 herself in front of a mirror without any aiding person.

1. A method for a volume adaptation of a breast prosthesis, wherein the breast prosthesis has a first shell body, a second shell body peripherally connected thereto, and a fluid space arranged between said shell bodies, wherein the fluid space is accessible from the outside through a passage provided with a valve, wherein the method comprises the following steps:
(a) inserting the breast prosthesis into a brassiere worn by a patient;
(b) filling the fluid space with a fluid or removing a fluid from the fluid space while the breast prosthesis is inserted into the brassiere worn by the patient.

2. The method in accordance with claim 1, wherein the fluid for volume adaptation is a gas.

3. The method in accordance with claim 1, wherein the fluid for volume adaptation is a liquid.

4. The method in accordance with claim 3, wherein the method furthermore includes a step of a crosslinking of the liquid filled into the fluid space.

5. The method in accordance with claim 1, wherein an additional medium is present within the fluid space that is different from the fluid for volume adaptation to be filled in step (b) and that reduces the adhesive tendency of the oppositely disposed inner surfaces of the shell bodies.

6. The method in accordance with claim 1, wherein the passage is a valve tube that extends in the radial direction through the connection region between the two shell bodies.

7. The method in accordance with claim 6, wherein a needle is placed into the valve tube to fill or empty the fluid space; and in that the fluid for volume adaptation is pressed through this needle into the fluid space.

8. The method in accordance with claim 7, wherein the valve tube comprises a step at its inner side at which its inner diameter reduces in the direction toward the fluid space.

9. The method in accordance with one of claim 1 wherein the breast prosthesis is connected to a filling and/or removal device between steps (a) and (b).

10. The method in accordance with claim 9, wherein the filling and/or removal device is connected to the brassiere and/or to the patient before it after it is connected to the breast prosthesis.

11. The method in accordance with claim 8, wherein the needle has a corresponding step at the outer side at which its inner diameter reduces in the direction toward the fluid space.

12. The method of claim 2, wherein the gas is air.

13. The method of claim 3, where in the liquid is a subsequently crosslinkable viscous liquid.
